(19) United States
(12) Patent Application Publication
(10) Pub. No.: US 2024/0307705 A1
(43) Pub. Date: Sep. 19, 2024

(54) SUBCUTANEOUS PHOTOBIOMODULATION

(71) Applicant: Case Western Reserve University, Cleveland, OH (US)

(72) Inventors: Michael MOFFITT, Cleveland, OH (US); Stephen LEWIS, Cleveland, OH (US); Michael JENKINS, Cleveland, OH (US)

(21) Appl. No.: 18/546,639

(22) PCT Filed: Feb. 21, 2022
(86) PCT No.: PCT/US2022/017173

§ 371 Date: Aug. 16, 2023

Related U.S. Application Data
(60) Provisional application No. 63/151,075, filed on Feb. 19, 2021.

(51) Int. Cl. A61N 5/06

(52) U.S. Cl. CPC A61N 5/0622; A61N 5/0601

(57) ABSTRACT

Subcutaneous photobiomodulation (PBM) is described for treatment of one or more medical conditions, including pulmonary fibrosis, an abnormality in upper airway function, hypertension, congestive heart failure, pulmonary hypertension, etc. The system includes a light source that can generate light inside the body (subcutaneously), a light transmission medium to transmit the light to an emitter, and the emitter sized and shaped to deliver the light directly to a target area associated with pulmonary fibrosis, an abnormality in upper airway function, hypertension, congestive heart failure, and/or pulmonary hypertension.

### CROSS-REFERENCE TO RELATED APPLICATIONS

This application claims the benefit of U.S. Provisional Application Ser. No. 63/151,075, filed 19 Feb. 2021, entitled “SUBCUTANEOUS PHOTOBIOMODULATION”, the entirety of which is incorporated by reference for all purposes.

### TECHNICAL FIELD

The present disclosure relates generally to photobiomodulation (PBM) and, more specifically, to treating a medical condition (e.g., pulmonary fibrosis, an abnormality in upper airway function, hypertension, congestive heart failure, pulmonary hypertension, or the like) with subcutaneous delivery of PBM directly to a target area related to the medical condition.

### BACKGROUND

Photobiomodulation (PBM) refers to the delivery of light to a target area in a patient's body at a specific dosing scheme (e.g., wavelength, power, time, etc.) to achieve non-thermal responses in the target area. However, due to delivery constraints associated with traditional transcutaneous delivery of the PBM, these theoretical varied non-thermal responses largely have not been achieved. With transcutaneous delivery, the light of PBM must travel through the patient's skin and other tissue layers of the patient's body, which can absorb the light of the PBM, to reach the target area. Additionally, the PBM source requires greater power for an adequate amount of light to reach the target area, often more power than is permitted for safe delivery of the PBM.

### SUMMARY

Subcutaneous photobiomodulation (PBM) can eliminate the delivery constraints and can allow the PBM to achieve closer to theoretical responses in a target area (it is believed that these responses are non-thermal). Accordingly, the responses of subcutaneous PBM can be used to treat a medical condition (e.g., pulmonary fibrosis, an abnormality in upper airway function, hypertension, congestive heart failure, pulmonary hypertension, etc.).

In an aspect, the present disclosure can include a system that can be used to treat pulmonary fibrosis, an abnormality in upper airway function, hypertension, congestive heart failure, or pulmonary hypertension. The system can include a light source configured to be implanted subcutaneously to a location within a patient's body (which may be a central location that can be easily accessible) to deliver a light signal according to a predefined dosing requirement to treat pulmonary fibrosis, an abnormality in upper airway function, hypertension, congestive heart failure, or pulmonary hypertension. In some instances, the predefined dosing requirement can be set by a medical professional to be unchangeable by the patient (e.g., a daily dose of light administrable by the patient). In other instances, the predefined dosing requirement can be a dose limit, such that the patient can administer as much light as desired up to the limit. Dosing can also be determined from sensing instrumentation in a feedback loop. The light source can include a non-transitory memory to store the predefined dosing requirement and a wireless transmitter to communicate with an external controller. The system can also include a light transmission mechanism configured to interface with the light source to transmit the light signal over a distance within the patient's body; and a transmitter configured to interface with the light transmission medium to deliver the light signal to a target within the patient's body to treat pulmonary fibrosis, an abnormality in upper airway function, hypertension, congestive heart failure, or pulmonary hypertension. The transmitter can be placed proximal to a target (which can be chosen based on pulmonary fibrosis, airway dysfunction, hypertension, congestive heart failure, or pulmonary hypertension) and may be sized and shaped based on a size and shape of the target.

In another aspect, the present disclosure can include a method for treating pulmonary fibrosis, an abnormality in upper airway function, hypertension, congestive heart failure, or pulmonary hypertension. The method can include delivering a predefined dosing requirement of a light signal (predefined based on a requirement for treating pulmonary fibrosis, an abnormality in upper airway function, hypertension, congestive heart failure, or pulmonary hypertension) by a subcutaneous light source within a patient's body to a light transmission mechanism within the patient's body (which may be a central location that can be easily accessible); transmitting the predefined dosing requirement of the light signal across a light transmission mechanism within the patient's body to an emitter within the patient's body (proximal to a target area for treating pulmonary fibrosis, an abnormality in upper airway function, hypertension, congestive heart failure, or pulmonary hypertension); and delivering the predefined dosing requirement of the light signal to a target within the patient's body. The emitter can be sized and shaped based on a size and shape of the target.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing an example of a system that can be used to deliver photobiomodulation (PBM) subcutaneously in accordance with an aspect of the present disclosure;

FIG. 2 is a diagram showing an example extension of the system shown in FIG. 1 to include an external programmer;

FIG. 3 is a diagram showing an example of how the systems of FIGS. 1 and 2 are used to deliver PBM subcutaneously;

FIG. 4 is a process flow diagram illustrating a method for subcutaneous PBM delivery in accordance with another aspect of the present disclosure;

FIG. 5 shows an example configuration of a fully implantable system to light to an affected lung to treat idiopathic pulmonary fibrosis; and

FIGS. 6 and 7 show example configurations of a fully implantable system to light to nerves of a neck region.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “photobiomodulation (PBM)” can refer to a form of light therapy based on the delivery of light with proper wavelengths to a patient at a specific dosing scheme to achieve a desired response (or effect) at a target area. PBM utilizes non-ionizing light sources, including lasers, light emitting diodes, and/or broadband light. In some examples, the light can have a wavelength between 250 and 1600 nm. However, as an example, the wavelength can be in the visible range (e.g., 400 nm-700 nm) and/or near-infrared range (e.g., 700 nm-1100 nm) of the electromagnetic spectrum.

As used herein, the term “subcutaneous” can refer to something that is made, done, or effected within a patient's body under the skin (anywhere in the intracorporeal region). A desired configuration for a stimulation can be determined/programmed in the extracorporeal region, while the stimulation can be configured according to the desired configuration and delivered in the intracorporeal region. It should be understood that subcutaneous refers to within a patient's body.

As used herein, the term “extracorporeal” can refer to something being outside a subject or patient's body (or, in other words, outside the skin).

As used herein, the term “intracorporeal” can refer to something being within the body (or, in other words, anywhere under the skin).

As used herein, the terms “target area” and “target location” can refer to a portion of a subject's body in need of PBM.

As used herein, the term “light pipe” can refer to a biocompatible elongated light transmission medium, such as one or more optical fibers or transparent plastic rods for transmitting light lengthwise through a patient's body.

As used herein, the term “dosing requirement” can refer to one or more characteristics of a dose for treating a medical condition.

As used herein, the terms “subject” and “patient” can be used interchangeably and refer to any warm-blooded organism including, but not limited to, a human being, a pig, a rat, a mouse, a dog, a cat, a goat, a sheep, a horse, a non-human primate, a rabbit, a cow, etc.

### II. Overview

In theory, photobiomodulation (PBM) can be used to achieve non-thermal effects or responses in the target area to treat a medical condition. Examples of these effects or responses can include nerve block, anti-inflammation (e.g., by activating anti-inflammatory microglia), anti-neurodegeneration (e.g., by overcoming cellular oxidative stress), anti-fibrotic responses in pathological fibrosis, improved cellular function (e.g., by improved cellular respiration), and the like. However, due to delivery constraints, including power constraints, and absorbing skin/intervening tissue, these theoretical varied non-thermal responses largely have not been achieved. It is well recognized that a greater effect may be achieved with lower power requirements if the light were to be applied directly to the target location (e.g., using a subcutaneous arrangement).

Subcutaneous arrangements that are fully implantable provide an alternative to traditional PBM delivery mechanisms, removing the absorbing skin and tissue layers that limit the effectiveness of PBM. The power is especially important when targeting small nerve fibers, while allowing other larger fibers to propagate normally, or similarly when targeting a very specific area of the brain or body. Specifically, the subcutaneous arrangements allow for treatment of medical conditions, like pulmonary fibrosis, an abnormality in upper airway function, hypertension, congestive heart failure, pulmonary hypertension, etc. (in other words, conditions affected by small nerve fibers or those requiring specific targeting).

### III. Systems

An aspect of the present disclosure relates to systems that can provide chronic or temporary photobiomodulation (PBM) to one or many target areas subcutaneously. PBM generally refers to the delivery of a dose of light with a proper wavelength (e.g., one or more predefined wavelengths between 600 nm and 1200 nm) at a specific dosing scheme to a target area or target location within the body to achieve a desired response. With PBM, different effects or responses can be achieved in different target areas or target locations, including nerve block, anti-inflammation (e.g., by activating anti-inflammatory microglia), anti-neurodegeneration (e.g., by overcoming cellular oxidative stress), anti-fibrotic responses in pathological fibrosis, improved cellular function (e.g., by improved cellular respiration), and the like.

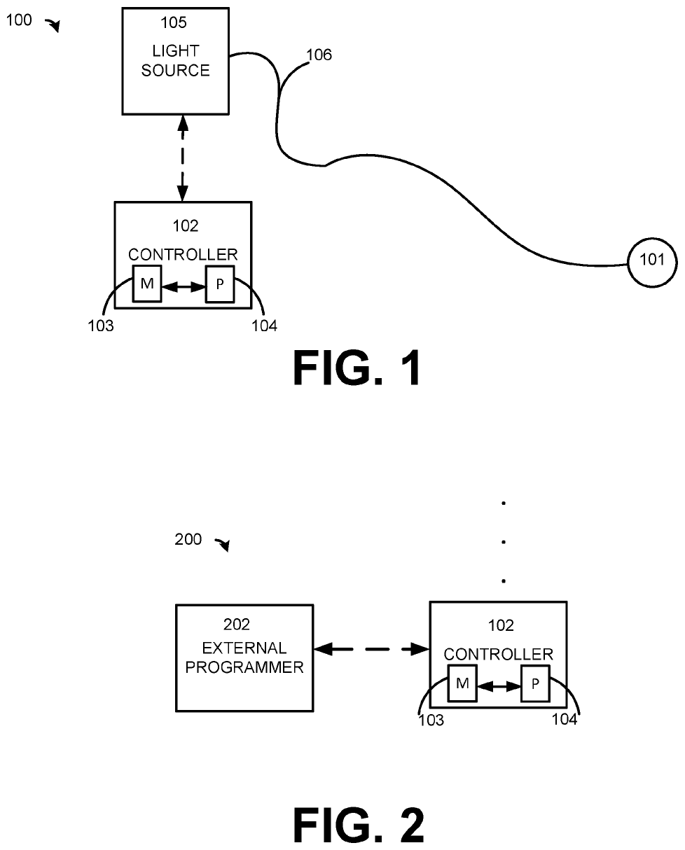

Shown in FIG. 1 is a system that can deliver the PBM to an associated target area subcutaneously (under the skin, where light is generated and delivered intracorporeally). Subcutaneous parts of the system include a light source 105, a light transmission mechanism (e.g., a light pipe 106) and an emitter 101. It should be appreciated that while the light pipe 106 is shown in the drawings and described herein, in some instances, the light source 105 can be a hermetic module at the end of an electrical lead, in which case the light pipe 106 is not needed.

The prescribed dose of light can be defined extracorporeally and delivered to the light source 105 by an external controller 102. The external controller 102 can establish a wireless connection with the light source 105 for data transfer (e.g., by inductive coupling, capacitive coupling, via low-energy Bluetooth, or the like). In some instances, the predefined dosing requirement can be set by a medical professional to be unchangeable by the patient (e.g., a daily dose of light administrable by the patient). In other instances, the predefined dosing requirement can be a dose limit, such that the patient can administer as much light as desired up to the limit. The light source can include a non-transitory memory to store the predefined dosing requirement and a wireless transmitter to communicate with an external controller.

The external controller 102 can include a non-transitory memory (M) and a processor (P). The light source 105 may also include a non-transitory memory, processor (which may be implemented as a microprocessor, a state machine, or the like), or other circuitry. It will be understood that the external controller 102 and/or the light source 105 can include additional hardware, such as a wireless transmitter that enables wireless communication with other devices, such as devices accessible within the cloud, devices associated with one or more clinicians, devices associated with the patient. In some instances, the external controller 102 can be battery powered. In other instances, the external controller 102 can receive line power. In still other instances, the external controller 102 can recharge the battery via line power. The external controller 102 can provide power to the light source 105—e.g., by establishing an RF connection between the external controller 102 and an element of the subcutaneous system. For example, the light source 105 can receive power from the controller 102 when coupled together. As another example, the light source 105 can be powered by battery power (which may be rechargeable by a connection to the external controller 102)—e.g., the battery may be located within an element of the subcutaneous system.

In the example shown in FIG. 1, the memory (M) 103 can store a predefined dose and the processor (P) 104 can access the memory (M) 103 and signal the light source 105 to generate a light signal for PBM of a target area within a patient's body based on the predefined dosing requirement. The predefined dosing requirement can include an optical power, a pulse width, a pulse shape, a frequency, an intensity, a cycling parameter comprising one or more period(s) of on time or off time, an amount of light delivered per unit time, a total amount of light to be delivered, or the like. In some instances, the light source 105 can include its own memory and/or processor. However, the light source 105 may be a slave to the controller and use the memory (M) 103 and/or processor (P) 104 of the controller. As an example, the light source 105 can include a laser, a laser diode, a light emitting diode, a broadband source, or the like that receives power either from its own power source or from a power source associated with the controller 102.

The predefined dosing requirement can be programmed by a clinician using a clinician programmer. In some instances, the patient may not be able to change the dose configured by the clinician directly; instead, the clinician must perform the changes (e.g., during a clinic visit, a virtual visit, or over a network, like the cloud, or the like). In other instances, the patient may be able to change the dose to a different value, as long as the value is within a window (e.g., between a lower limit and an upper limit) that has been prescribed/preset by the clinician. In other instances, sensing instrumentation can feed information back to determine dosing,

In the example shown in FIG. 1, external controller 102 can signal the light source 105 to generate a light signal for PBM of a target area within a patient's body based on the predefined dosing requirement. The predefined dosing requirement can include an optical power, a pulse width, a pulse shape, a frequency, an intensity, a cycling parameter comprising one or more period(s) of on time or off time, an amount of light delivered per unit time, a total amount of light to be delivered, or the like. In some instances, the light source 105 can include its own memory and/or processor. However, the light source 105 may be a slave to the external controller. As an example, the light source 105 can include a laser, a laser diode, a light emitting diode, a broadband source, or the like that receives power either from its own power source or from a power source associated with the external controller 102.

The predefined dosing requirement can be programmed by a clinician using a clinician programmer. In some instances, the patient may not be able to change the dose configured by the clinician directly; instead, the clinician must perform the changes (e.g. during a clinic visit, a virtual visit, or over a network, like the cloud, or the like). In other instances, the patient may be able to change the dose to a different value, as long as the value is within a window (e.g., between a lower limit and an upper limit) that has been prescribed/preset by the clinician. In other instances, sensing information can be fed back to determine the dosing.

The light source 105 can generate the light signal according to the predefined dosing requirement and send the light signal through a light transmission mechanism 106 (also referred to as a light pipe, which may include an optical pipe and electrical wires) to deliver the light signal to an emitter 101 to deliver the light signal to a target area or target location within the patient's body. The emitter 101 can be shaped and sized based on the target location. In some instances, the light source 105 can be associated with a unique identifier, such as an RFID, or an identifier stored in non-volatile memory and accessible by an extracorporeal device, which can prevent a patient from using another patient's preprogrammed controller.

As shown in FIG. 2, the external controller 102 can be in wireless communication with an external programmer 202. Although illustrated as wireless communication, it will be understood that the controller 102 can engage in wired communication with the external programmer 202. The external programmer 202 can be one or more computing devices that may be remotely or locally located with respect to the controller 102. In some instances, the controller 102 and the external programmer 202 can be connected through the cloud and each can use the cloud to store data and instructions. In other instances, the controller 102 can include the external programmer 202. As an example, the single device can include a clinician mode for programming by a clinician.

The external programmer 202 can provide or edit at least one aspect of the predefined dosing requirement used for PBM (e.g., dose parameters, total amount of light to be received by the patient, in a time, such as a day, week, month, 3 months, 6 months, 9 months, year, etc.). The external programmer 202 can also create a link between the controller 104, the light source 105. For example, the external programmer 202 can be a clinician programmer that resides in a clinician's office and can be used to set or edit the predefined dosing requirement, such as setting one or more optical dose parameters or defining a therapy program. The external programmer 202 can also receive communication from the controller 102 regarding progress of the patient using the PBM. For example, the controller 102 can track the amount of light that is or has been delivered to the patient over a period of time and this information can be transmitted to the external programmer 202. As an example, the therapy program can be stored in the cloud with a local copy stored in the memory (M) 103 of the controller so that the patient does not have to have the controller 102 connected to the internet to use the therapy program. As another example, the controller 102 can communicate with a device associated with the patient and convey pertinent information, such as the amount of therapy remaining on a prescription, the state of the batteries of the controller 102, illumination parameters, program usage data, or the like. In another example, the controller can receive data from a device associated with the patient including patient diary data, activity data, heart rate, physician indicated task, other health-related data or the like. The controller 102 can aggregate the data in the cloud and make the data accessible to the external programmer 202.

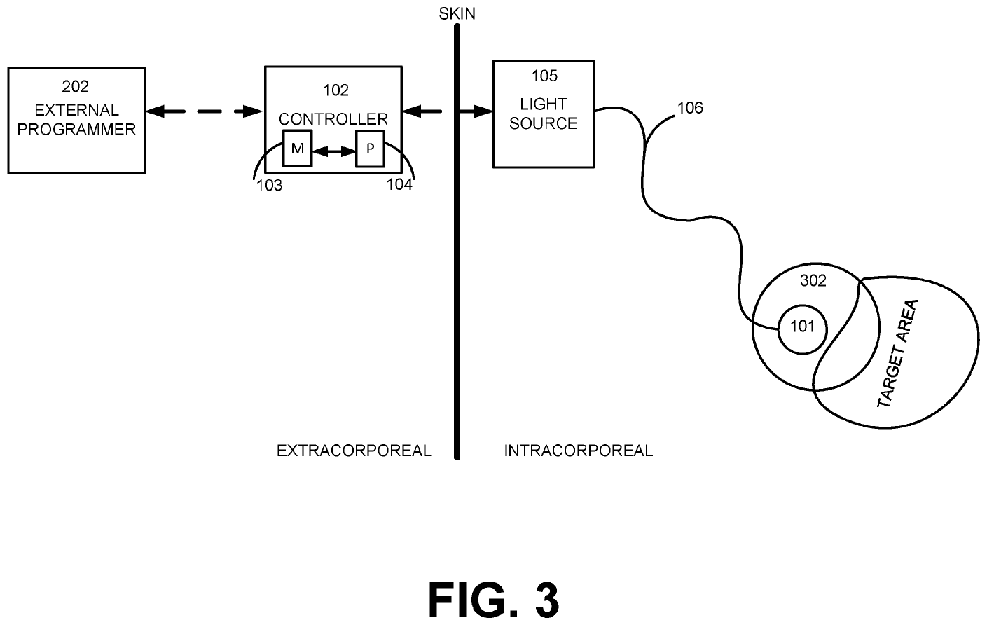

As shown in FIG. 3, the system can deliver information related to a predefined dosing requirement through a portion of the patient's skin, to the light source 105, which generates light and allows light to travel into the patient's body. The external controller 102 and additional components, like an external programmer 202, can be within the extracorporeal portion of the system (in other words, outside of the body). The light source 105, light pipe 106 and the light delivery element (emitter 101) can also be within the intracorporeal portion of the system. The emitter 101 can deliver the light signal 302 to the target area within the patient's body. The emitter 101 can be shaped and sized according to the target area. The emitter 101, which can be sized and shaped according to the target area, can deliver an amount of light to the target area within the patient's body. It should be understood that the target area can be suppressed, while other nearby cells should not be activated or suppressed.

### IV. Methods

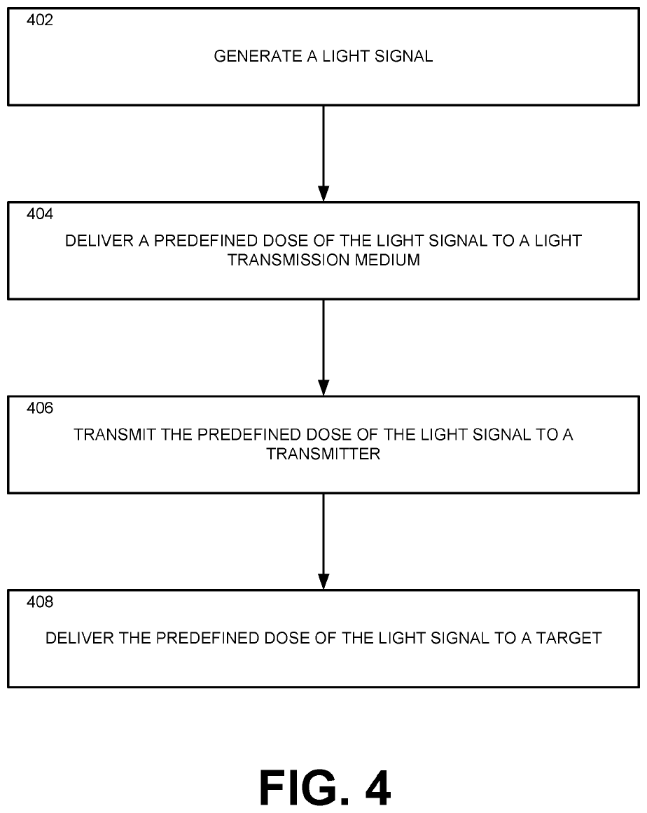

Another aspect of the present disclosure can include a method for using a system (shown in FIGS. 1-3, for example) for subcutaneous photobiomodulation (PBM), as shown in FIG. 4. In most basic form, the system can include an external controller 102 in wireless communication (through the patient's skin) with a light source 105 that generates a light signal that is delivered to a target area (e.g., through a light pipe 106 with final delivery by an emitter 101, for example). The external controller 102 can also be in wireless communication with one or more external devices (e.g., an external programmer device 202). Steps of the method can be performed by the external controller 102 that includes a memory storing a predefined dosing requirement and a processor configured to access the memory and signal the light source 105 to generate a light signal for PBM of a target area within a patient's body based on the predefined dosing requirement. As an example, the external programmer device 202 can edit one or more aspects of the predefined dosing requirement.

For purposes of simplicity, the method is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method, nor is the method necessarily limited to the illustrated aspects. Additionally, at least the external controller 102 is a computer-related entity that includes hardware, including a memory (which is a non-transitory memory) and a processor (e.g., a microprocessor, a state machine, or the like, and communicates with hardware (e.g., light source 105 and external programmer device) to facilitate the performance of subcutaneous PBM. Moreover, the light source 105 can receive power from the external controller 102 and/or may have an internal power source, like a battery.

A light signal can be generated (e.g., by a subcutaneous light source 105) according to a predefined dosing requirement (based on the medical condition). At Step 202, the predefined dosing requirement (e.g., an optical power, a pulse width, a pulse shape, a frequency, an intensity, a cycling parameter comprising a period of on time or off time, an amount of light delivered per unit time, a total amount of light to be delivered, etc., established by controller 102; the predefined dosing requirement can be received and/or edited based on instructions received wirelessly from a device associated with an external programmer device) of the light signal can be delivered to a light transmission medium (e.g., light transmission medium 106). At Step 204, the predefined dosing requirement of the light signal can be transmitted to an emitter (e.g., emitter 101). At Step 206, the predefined dosing requirement of the light signal can be delivered to the target (e.g., by the emitter 101) related to the medical condition to treat the medical condition.

It should be noted that the external controller 102 can log information related to the light signal, including the number of doses given to the patient. For example, based on the number of doses given to the patient, the controller 102 can communicate wirelessly with a device associated with a doctor and the doctor can evaluate the usage and/or alter the predefined dosing requirement.

### V. Example Medical Conditions

Subcutaneous photobiomodulation (PBM) provides a focused delivery of light that can be used to treat medical conditions requiring specific targeting or those affected by small nerve fibers that would not be treatable with traditional transcutaneous PBM.

### Medical Conditions Requiring Specific Targeting

An example of a medical condition requiring specific targeting is pulmonary fibrosis (also referred to as idiopathic pulmonary fibrosis or IPF). Pulmonary fibrosis is a lung disease that occurs when lung tissue becomes damaged and scarred so that the lung tissue thickens and stiffens. With pulmonary fibrosis, fibrosis encroaches on normal pulmonary tissue such that breathing is progressively compromised until death a few years after diagnosis. Currently, no effective treatments exist for pulmonary fibrosis, but data from a mouse model suggests that PBM can slow or inhibit progression of the pulmonary fibrosis. The mouse model has made PBM attractive to use for human treatment of pulmonary fibrosis, but traditional transcutaneous PBM is ineffective. However, a subcutaneous solution would be able to specifically target areas to slow or inhibit progression of pulmonary fibrosis. Other conditions that can be treated in the brain, for example, are ALS, progressive supranuclear palsy (PSP), and Huntington's disease.

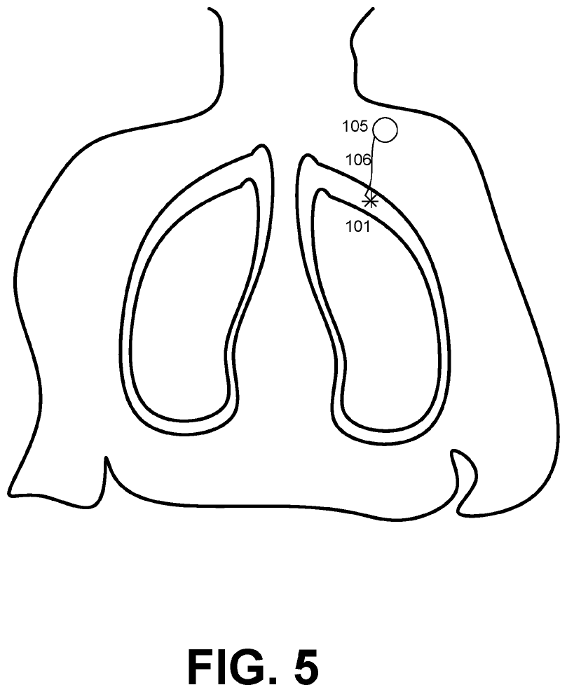

Referring to FIG. 1 (also shown in FIG. 5), the light source 105 can be a subcutaneous light source that is placed a location within a patient's body (at least under the patient's skin). The light source 105 can generate light according to a predefined dosing requirement (which may include a certain delivery characteristic—at least one of an optical power, a pulse width, a frequency, an intensity, a cycling parameter comprising one or more period(s) of on time or off time, an amount of light delivered per unit time, a total amount of light to be delivered-defined based on the patient, an average of similar patients, a tolerance of the patient, a position of emitter 101, and/or the progression of the disease). The predetermined dose can be provided and/or edited by external controller 102 and stored in a non-transitory memory associated with the light source 105. Accordingly, the light source 105 can include a wireless transmitter, such as a RF coil or a magnet. The predefined dosing requirement can be transmitted through the patient's body using a light pipe 106. The light source 105 can be positioned considering the size and/or length of the light pipe 106; in many cases, it is important to minimize at least the size and/or length of the light pipe 106. Accordingly, the light source 105 can be implanted in the sub-clavicular upper chest region (this region has the additional advantage that clinicians are accustomed to placing devices such as pacemakers, implantable defibrillators, and DBS devices in this region).

The light source 105 can be powered by an associated battery (that may be implanted with the light source 105) and/or by an RF communication between the light source 105 and the external controller 102 and/or a device associated with the external controller 102. When the RF communication is used, the external RF power source (the external controller 102 and/or a device associated with the external controller 102). As an example, the external RF power source that is positioned over the light source 105 to deliver power when therapy is delivered.

The light pipe 105 can transmit the predefined dosing requirement of the light signal through the patient's body to an emitter 101. The emitter 101 can be at a location surrounding and/or within the lung. For example, the emitter 101 can be positioned at or near the top of the lung or the bottom of the lung. The emitter 101 can be located at a location surrounding and/or within the lung. The emitter 101 can deliver the predefined dosing requirement of the light signal to a target within the patient's lung to slow or inhibit progression of the pulmonary fibrosis. The steps of delivering the light subcutaneously can be repeated one or more of several times a day, daily, or weekly.

Medical Conditions affected by Small Nerve Fibers

Subcutaneous delivery of PBM can be used to target small nerve fibers (e.g., afferent C-fibers), while large nerve fibers can be allowed to conduct. Examples of medical conditions affected by small nerve fibers include an abnormality in upper airway function, hypertension, congestive heart failure, and pulmonary hypertension.

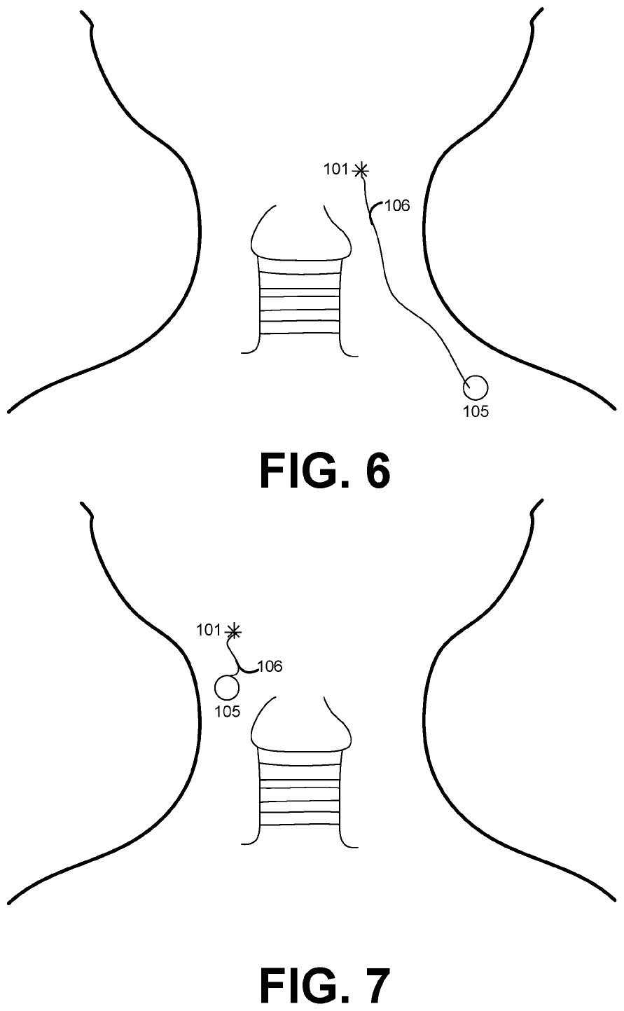

### Abnormality in Upper Airway Function (Shown in FIGS. 6 and 7)

An example of a medical condition affected by small nerve fibers includes an abnormality in upper airway (e.g., a tongue, a larynx, and/or a nasopharynx) function. Such an abnormality in upper airway function can be treated by (referring to FIG. 1) delivering a predefined dosing requirement (e.g., an optical power, a pulse width, a frequency, an intensity, a cycling parameter comprising one or more period(s) of on time or off time, an amount of light delivered per unit time, a total amount of light to be delivered) of a light signal by a subcutaneous light source 105 to a light pipe 106; transmitting the predefined dosing requirement of the light signal across the light pipe 106 through the patient's body to an emitter 101; and delivering the predefined dosing requirement of the light signal to slow or inhibit afferent conduction of at least one small peripheral nerve fiber in the at least one peripheral nerve by the emitter 101 to treat the abnormality in upper airway function. The light source 105 can be located in a patient's neck or in a sub-clavicular upper chest region of the patient's body (the location may be chosen to minimize the size/length of the light pipe), while the emitter 101 can be located proximal to at least one peripheral nerve. The emitter can be located/shaped/sized based on a size or a shape of the at least one of the small peripheral nerve fibers within the at least one peripheral nerve. The small peripheral nerve fibers can be afferent small diameter fibers (e.g., smaller than A-beta fibers, like C-fibers), which can be suppressed, while other larger fibers (e.g., A-beta fibers) can be allowed to continue to transmit action potentials normally. For example, suppression of small diameter afferents in the superior laryngeal nerve (main branch and internal and external branches) and inferior laryngeal nerves can eliminate abnormal upper airway reactivity (e.g., excessive cough, reactivity to ingested substances and cold air) from the upper airway including the tongue, larynx and nasopharynx, while suppression of small diameter afferents in recurrent laryngeal nerves can eliminate abnormal upper airway reactivity (e.g., excessive cough, reactivity to ingested substances and cold air) from the upper airway including the larynx and lower nasopharynx. Suppression of small diameter afferents in glossopharyngeal nerves can eliminate upper airway obstruction caused by abnormal positioning of the tongue, as well as closure of the nasopharynx (e.g., to treat obstructive sleep apnea). Other treatments for obstructive sleep apnea include suppression of small diameter afferents in superior laryngeal nerves (main branch or internal/external branches) can eliminate upper airway obstruction caused by closure of the nasopharynx and larynx, and suppression of small diameter afferents in the inferior pharyngeal nerves can eliminate upper airway obstruction caused by closure of the nasopharynx.

As an example, the light source 105 can be small enough to be located in the neck, which has the advantage of reduced fatigue of the light pipe 106 by spanning less of the neck joint. A fixation device can be used to hold the light pipe 106 and/or the light emitter 101 in position near to a target nerve or tissue. One possible fixation device is a sleeve that is attached to the light pipe and/or the light emitter with controlled force, and that is sutured to adjacent tissue such as fascia. As previously described, the light source 105 can include an implantable battery or may not have a battery but instead may be powered by an external RF power source that is positioned over the light source 105 when therapy is delivered (e.g., a few times a day, daily, weekly, etc.).

### Hypertension, Congestive Heart Failure, Pulmonary Hypertension

Other examples of medical conditions affected by small nerve fibers includes hypertension, congestive heart failure, and pulmonary hypertension. Similarly to what is described above, each of hypertension, congestive heart failure, and pulmonary hypertension can be treated by (referring to FIG. 1) delivering a predefined dosing requirement (e.g., an optical power, a pulse width, a frequency, an intensity, a cycling parameter comprising one or more period(s) of on time or off time, an amount of light delivered per unit time, a total amount of light to be delivered) of a light signal by a subcutaneous light source 105 to a light pipe 106; transmitting the predefined dosing requirement of the light signal across the light pipe 106 through the patient's body to an emitter 101; and delivering the predefined dosing requirement of the light signal to slow or inhibit afferent conduction of at least one small peripheral nerve fiber in the at least one peripheral nerve by the emitter 101 to treat the hypertension, congestive heart failure, or pulmonary hypertension. The light source 105 can be located in a sub-clavicular upper chest region of the patient's body (the location may be chosen to minimize the size/length of the light pipe), while the emitter 101 can be located proximal to at least one peripheral nerve. The emitter can be located/shaped/sized based on a size or a shape of the at least one of the small peripheral nerve fibers within the at least one peripheral nerve. The small peripheral nerve fibers can be afferent small diameter fibers, which can be suppressed, while other larger fibers can be allowed to continue to transmit action potentials normally. For example, suppression of small diameter C-fiber afferents in the carotid sinus nerve can be suppressed to eliminate the development of hypertension; small diameter C-fiber afferents in the carotid sinus nerve can be suppressed to eliminate breathing disturbances associated with disease processes, such as hypertension, small diameter C-fiber afferents in the cardiac nerve can be suppressed to eliminate cardiac arrhythmias with disease processes such as congestive heart failure and pulmonary hypertension. Additionally, suppression of a small fiber afferent in the renal nerve and associated system to induce a renal deafferentation to treat hypertension. For example, hypertension may be caused by chronic kidney disease or other disease states, and treatable with renal deafferentation with PBM.

As previously described, the light source 105 can include an implantable battery or may not have a battery but instead may be powered by an external RF power source that is positioned over the light source 105 when therapy is delivered (e.g., a few times a day, daily, weekly, etc.).

From the above description, those skilled in the art will perceive improvements, changes, and modifications. Such improvements, changes and modifications are within the skill of one in the art and are intended to be covered by the appended claims.

The following is claimed:

1. A method for treating pulmonary fibrosis, the method comprising:
delivering a predefined dosing requirement of a light signal by a subcutaneous light source;
transmitting the predefined dosing requirement of the light signal through the patient's body to an emitter, wherein the emitter is located at a location proximal to and/or within the lung;
delivering the predefined dosing requirement of the light signal to a target at the patient's lung by the emitter to slow or inhibit progression of the pulmonary fibrosis.

2. The method of claim 1, further comprising repeating the delivering, transmitting, and delivering one or more of several times a day, daily, or weekly.

3. The method of claim 1, further comprising powering the subcutaneous light source by an external RF power source that is positioned over an RF receiver that is part of the subcutaneous system.

4. The method of claim 1, further comprising using a battery within the subcutaneous system to power the subcutaneous light source.

5. The method of claim 1, further comprising configuring the predefined dosing requirement with at least one delivery characteristic based on a level of disease in the patient, a patient tolerance, and/or a position of the emitter.

6. The method of claim 1, wherein the predefined dosing requirement comprises at least one of an optical power, a pulse width, a pulse shape, a frequency, an intensity, a cycling parameter comprising one or more period(s) of on time or off time, an amount of light delivered per unit time, a total amount of light to be delivered.

7. The method of claim 1, wherein the subcutaneous light source comprises a wireless transmitter to communicate with an external device to receive updates to the predefined dosing requirement.

8. The method of claim 1, wherein the subcutaneous light source comprises a non-transitory memory to store the predefined dosing requirement.

9. A method for treating an abnormality in upper airway function, the method comprising:
delivering a predefined dosing requirement of a light signal by a subcutaneous light source;
transmitting the predefined dosing requirement of the light through the patient's body to an emitter, wherein the emitter is located proximal to at least one peripheral nerve;
delivering the predefined dosing requirement of the light signal to slow or inhibit afferent conduction of at least one small peripheral nerve fiber in the at least one peripheral nerve by the emitter to treat the abnormality in upper airway function.

10. The method of claim 9, wherein the emitter is sized and/or shaped based one a size and/or shape of the at least one small peripheral nerve fiber.

11. The method of claim 9, wherein the at least one small peripheral nerve fiber is suppressed while other larger fibers are allowed to continue transmitting action potentials.

12. The method of claim 9, wherein the at least one peripheral nerve is a superior laryngeal nerve, an inferior laryngeal nerve, or a recurrent laryngeal nerve; and the at least one small peripheral nerve fiber is a small diameter afferent fiber to suppress at least one of excessive cough, reactivity to an ingested substance, and reactivity to cold air.

13. The method of claim 12, wherein the upper airway comprises a tongue, a larynx, or a nasopharynx.

14. The method of claim 9, further comprising using a fixation device to hold the light pipe in position proximal to at least one peripheral nerve to interface with the emitter.

15. The method of claim 14, wherein the fixation device comprises a sleeve that is attached to a light pipe and/or the light emitter with a controlled force and is sutured to adjacent tissue.

16. A method for treating hypertension, congestive heart failure, or pulmonary hypertension, the method comprising:
delivering a predefined dosing requirement of a light signal by a subcutaneous light source;
transmitting the predefined dosing requirement of the light signal through the patient's body to an emitter, wherein the emitter is located proximal to a nerve of the patient;
delivering the predefined dosing requirement of the light signal to slow or inhibit afferent conduction of at least one small peripheral nerve fiber in a larger nerve by the emitter to treat hypertension, congestive heart failure, or pulmonary hypertension, wherein the larger nerve comprises a renal nerve, a carotid sinus nerve, a vagus nerve, or a cardiac nerve.

17. The method of claim 16, wherein the at least one small peripheral nerve fiber is suppressed while other larger fibers are allowed to continue transmitting action potentials.

18. The method of claim 17, wherein the at least one small peripheral nerve fiber is a small diameter afferent fiber.

19. The method of claim 16, wherein the emitter is sized and/or shaped based one a size and/or shape of the at least one small peripheral nerve fiber.

20. The method of claim 16, further comprising establishing a wireless communication between the subcutaneous system and an external device to update the predefined dosing requirement.
